(19) United States
(12) Patent Application Publication
(10) Pub. No.: US 2014/0087432 A1
(43) Pub. Date: Mar. 27, 2014

(54) METHODS FOR CONDITIONING PRETREATED BIOMASS

(71) Applicant: ABENGOA BIOENERGY, Chesterfield, MO (US)

(72) Inventors: Quang A. Nguyen, Chesterfield, MO (US); Leroy D. Holmes, Florissant, MO (US); James F. Schelert, Lincoln, NE (US); Mark A. Borchers, Valley Park, MO (US)

(73) Assignee: ABENGOA BIOENERGY, Chesterfield, MO (US)

(21) Appl. No.: 13/625,529

(22) Filed: Sep. 24, 2012

(51) Int. Cl. C12P 19/14

(52) U.S. Cl. CPC C12P 19/14
USPC 435/99; 165/104.11

(57) ABSTRACT

Methods for producing ethanol from cellulosic biomass and, in particular, methods for conditioning pretreated biomass are disclosed. In some embodiments, pretreated biomass is contacted with a cooling fluid in a flash vessel to cool the biomass. The amount of alkaline solution contacted with the biomass may be based on the pH of partially hydrolyzed pretreated biomass in a liquefaction bioreactor.

### FIELD OF THE DISCLOSURE

The field of the disclosure relates to methods for producing ethanol from cellulosic biomass and, in particular, methods for conditioning pretreated biomass. In some particular embodiments, pretreated biomass is contacted with a cooling fluid in a flash vessel to cool the biomass. Alternatively or in addition, the amount of alkaline solution contacted with the biomass is based on the pH of partially hydrolyzed pretreated biomass in a liquefaction bioreactor.

### BACKGROUND

A number of biofuels including ethanol have seen increased use as an additive or replacement for petroleum-based fuels such as gasoline. Ethanol may be produced by fermentation of simple sugars produced from sources of starch (e.g., corn starch) or from lignocellulosic biomass.

There are a variety of widely available sources of lignocellulosic biomass including, for example, corn stover, agricultural residues (e.g., straw, corn cobs, etc.), woody materials, energy crops (e.g., sorghum, poplar, etc.), and bagasse (e.g., sugarcane). Lignocellulosic biomass is a relatively inexpensive and readily available feedstock for the preparation of sugars, which may be fermented to produce alcohols such as ethanol.

Preparation of ethanol from biomass involves methods for increasing the accessibility of cellulose to downstream enzymatic hydrolysis (i.e., “pretreatment” operations). Before being subjected to such hydrolysis, the pretreated biomass may be conditioned for enzymatic hydrolysis to promote formation of simple sugars during hydrolysis.

This section is intended to introduce the reader to various aspects of art that may be related to various aspects of the disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY

One aspect of the present disclosure is directed to a method for conditioning pretreated biomass. Biomass is discharged from a pretreatment digester to a flash vessel to flash vapor from the biomass, the biomass forming a bed in the flash vessel. A cooling fluid is introduced into the flash vessel to cool the biomass. The cooling fluid has a temperature less than about 25° C.

Another aspect of the present disclosure is directed to a method for conditioning pretreated biomass. Pretreated biomass is discharged from a pretreatment digester. The pretreated biomass discharged from the digester is introduced into a neutralization mixer. An alkaline solution is introduced into the neutralization mixer to adjust the pH of the biomass. Enzyme is added to the pH-adjusted pretreated biomass. The enzyme-containing pretreated biomass is introduced into a liquefaction bioreactor to partially hydrolyze the pretreated biomass. The pH of the partially hydrolyzed pretreated biomass in the liquefaction bioreactor is measured. At least one of (1) the amount of alkaline solution introduced into the neutralization mixer and (2) the concentration of alkali in the alkaline solution is adjusted based on the measured pH of the partially hydrolyzed pretreated biomass in the liquefaction bioreactor.

Yet a further aspect of the present disclosure is directed to a method for conditioning pretreated biomass. Pretreated biomass is discharged from a pretreatment digester into a flash vessel. An alkaline solution is introduced into the flash vessel to adjust the pH of the biomass. Enzyme is added to the pH-adjusted pretreated biomass. The enzyme-containing pretreated biomass is introduced into a liquefaction bioreactor to partially hydrolyze the pretreated biomass. The pH of the partially hydrolyzed pretreated biomass in the liquefaction bioreactor is measured. At least one of (1) the amount of alkaline solution introduced into the flash vessel and (2) the concentration of alkali in the alkaline solution is adjusted based on the measured pH of the partially hydrolyzed pretreated biomass in the liquefaction bioreactor.

Various refinements exist of the features noted in relation to the above-mentioned aspects of the present disclosure. Further features may also be incorporated in the above-mentioned aspects of the present disclosure as well. These refinements and additional features may exist individually or in any combination. For instance, various features discussed below in relation to any of the illustrated embodiments of the present disclosure may be incorporated into any of the above-described aspects of the present disclosure, alone or in any combination.

### BRIEF DESCRIPTION OF THE DRAWINGS

FIG. 1 is a flow chart depicting a method for producing ethanol from a cellulosic biomass feedstock;

FIG. 2 is a schematic view of a system for pretreating biomass by steam explosion;

FIG. 3 is a side view of a flash vessel for flashing steam from biomass; and

FIG. 4 is a schematic view of pretreated biomass conditioning, enzyme mixing and liquefaction operations.

Corresponding reference characters indicate corresponding parts throughout the drawings.

### DETAILED DESCRIPTION

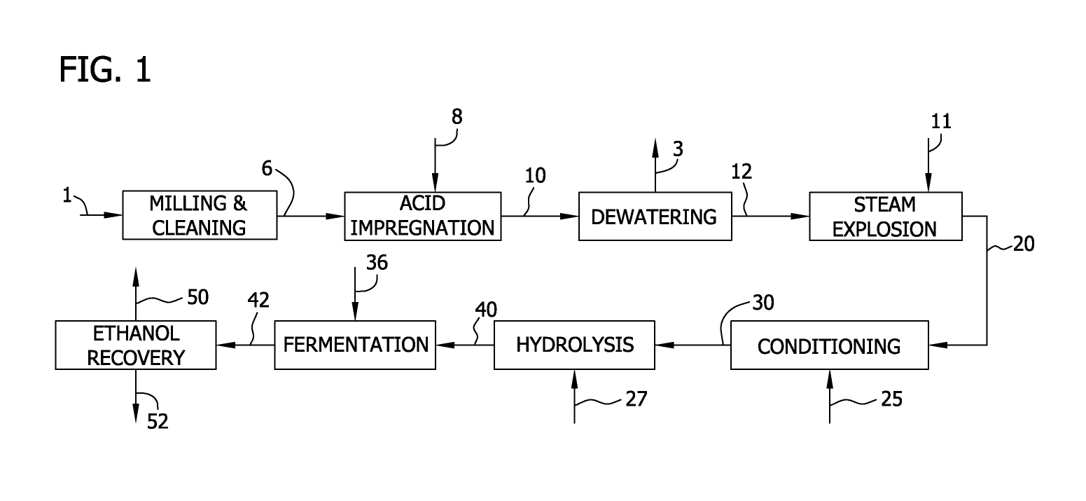

In accordance with various embodiments of the present disclosure and with reference to FIG. 1, lignocellulosic biomass material 1 is subjected to milling and cleaning operations to reduce the particle size of the material and to remove any non-biomass contaminants from the feedstock. Any of a variety of biomass materials may be used as the starting feedstock of embodiments of the present disclosure including plant biomass, agricultural or forestry residues, or sugar processing residues. Suitable grass materials include cord grass, reed canary grass, clover, switchgrass, bamboo, marram grass, meadow grass, reed, ryegrass, sugar cane, and grasses from the Miscanthus genus. The biomass feedstock may include agricultural residues such as rice straw, rice hulls, barley straw, corn cobs, wheat straw, canola straw, oat straw, oat hulls, corn fiber, stover (e.g., sorghum, soybean stover and/or corn stover) or combinations thereof. Sugar processing residues include sugar cane bagasse, sweet sorghum, beet pulp, and combinations thereof. The feedstock may also include wood and forestry wastes such as, for example, recycled wood pulp fiber, sawdust, hardwood, softwood, forest thinnings, orchard thinnings, or combinations thereof. Other materials such as residential yard waste, wood debris from construction and demolition sites and cellulosic materials sorted from municipal wastes may also be used in the feedstock. The content of such municipal wastes may vary (e.g., from about 15 wt % to about 50 wt % cellulose on a dry basis, from about 5 wt % to about 30 wt % hemicellulose on a dry basis and/or from about 10 wt % to about 40 wt % lignin on a dry basis).

The biomass feedstock may have a cellulose content of at least about 15 wt % on a dry basis or, as in other embodiments, at least about 25 wt %, at least about 30 wt %, at least about 35 wt % or at least about 50 wt % cellulose on a dry basis (e.g., from about 25 wt % to about 55 wt % or from about 30 wt % to about 45 wt %). Alternatively or in addition, the biomass feedstock may contain at least about 10 wt % hemicellulose on a dry basis or at least about 15 wt %, at least about 20 wt % or at least about 25 wt % hemicellulose on a dry basis (e.g., from about 10 wt % to about 40 wt % or from about 20 wt % to about 30 wt %). Alternatively or in addition, the biomass material may include at least about 10 wt % lignin on a dry basis or at least about 15 wt %, at least about 20 wt % or at least about 25 wt % lignin on a dry basis (e.g., from about 10 wt % to about 40 wt % or from about 15 wt % to about 25 wt %). In this regard, the biomass feedstock may contain cellulose, hemicellulose and/or lignin in any range bound by the above-listed parameters and in any combination of respective ranges. The biomass material may be bound by any combination of the above-noted parameters including any combination of the cellulose, hemicellulose and lignin parameters provided above. It should be noted that the recited ranges are exemplary and the biomass feedstock may contain more or less cellulose, hemicellulose and/or lignin without limitation. Any biomass material suitable for preparing fermentable sugars may be used unless stated otherwise.

The feedstock may include components other than cellulose, hemicellulose and lignin such as ash including structural inorganics and may include contaminants (e.g., gravel, sand or dirt). In various embodiments, the biomass feedstock may contain about 1 wt % or less ash on a dry basis, about 3 wt % or less ash, about 5 wt % or less ash or about 8 wt % or less ash on a dry basis. The biomass feedstock may contain moisture and in some embodiments contains at least about 1 wt % (by total weight including moisture) moisture, at least about 5 wt %, at least about 10 wt %, at least about 15 wt % or even at least about 20 wt % moisture (e.g., from about 1 wt % to about 30 wt %, from about 1 wt % to about 20 wt % or from about 5 wt % to about 20 wt % moisture).

The biomass feedstock material may undergo one or more milling operations to reduce the particle size of the material before downstream processing. In some embodiments, the biomass material 1 is reduced to a size less than about 40 mm or from about 2 mm to about 30 mm or from about 5 mm to about 30 mm. Relatively large biomass material (e.g., greater than about 40 mm or greater than about 50 mm) may result in low bulk density which increases the size of equipment (e.g., conveyors) and may impede impregnation and heating. Relatively small biomass (e.g., less than about 2 mm or less than about 0.5 mm) may hold large amounts of liquid resulting in longer heating times. Any equipment suitable to reduce the particle size of the biomass material 1 may be used including, for example, hammermills, grinders, cutters, chippers, crushers and the like. In some embodiments, the biomass feedstock is not milled prior to downstream processing.

Alternatively or in addition, the biomass feedstock may undergo a cleaning operation to remove contaminants from the feedstock. Suitable operations include sifting, air classifying to remove gravel, sand and fines, and contacting the feedstock with one or more magnets to remove ferrous material from the feedstock.

In some embodiments, the milled and cleaned biomass feedstock is preheated with direct steam contact (e.g., less than 1 bar pressure) to open up the pore structure and drive out entrapped air before feeding the biomass to the acid impregnator as described below. The steaming time may be sufficient to heat the biomass to at least about 40° C., at least about 60° C. or at least about 80° C.

After milling, the milled biomass 6 is subjected to an acid impregnation process and steam explosion process to cause the cellulose in the biomass to become more available to enzymatic hydrolysis. Acid impregnation generally involves contacting the milled biomass with acid (e.g., dilute acid) in a vessel for a time sufficient to allow the acid to thoroughly contact and be dispersed throughout the biomass. Any suitable vessel may be used to achieve acid impregnation including pug mixers and stirred tank reactors that may be operated in batch or continuous modes. The biomass may be contacted with acid 8 by spraying and mixing or by soaking and mixing. In embodiments in which spraying and mixing are used to impregnate acid into the biomass, the liquid-to-dry biomass weight ratio may be at least about 2:1, at least about 3:1 or at least about 4:1 (e.g., from about 3:1 to 8:1). In embodiments in which the biomass is soaked and mixed, the liquid-to-dry biomass weight ratio may be at least about 10:1, at least about 12:1 or at least about 14:1 (e.g., from about 12:1 to about 20:1).

The acid 8 that is used for acid impregnation may be sulfuric acid, hydrochloric acid or nitric acid. Regardless of the acid that is used, the concentration of the acid solution added to the biomass may be at least about 0.1 wt %, at least about 0.5 wt %, at least about 1 wt %, at least about 2.5 wt %, less than about 5 wt %, less than about 3 wt %, less than about 2.5 wt %, less than about 1 wt % or less than about 0.5 wt % (e.g., from about 0.2 wt % to about 5 wt % or from about 0.5 wt % to about 3 wt %). The temperature of the acid 8 introduced in the vessel may vary depending on whether the acid-impregnation vessel includes heating elements (resistance heaters, combusted gases, steam or the like) in thermal communication with the vessel or includes direct steam injection for heating the acid and/or milled biomass material 6 during impregnation.

In some embodiments, the acid 8 is heated and/or extraneous heat is applied to the impregnation vessel such that the acid-impregnated biomass 10 is at a temperature of at least about 20° C., at least about 50° C. or at least about 75° C. The amount of time between initial contact of the biomass with acid and before downstream dewatering may be at least about 30 seconds, at least about 1 minute, at least about 5 minutes or more (e.g., from about 30 seconds to about 20 minutes or from about 1 minute to about 10 minutes). The pH of the acid-impregnated biomass 10 may be less than about 5, less than about 3 or less than about 2.

After acid impregnation, the acid-impregnated biomass 10 may undergo a dewatering operation (FIG. 1) to reduce the moisture content of the biomass to an amount suitable for steam explosion.

Suitable equipment for dewatering includes, for example centrifuges, filters and cyclones (e.g., which may also be referred to as “hydro-clones” by those of skill in the art) which may be used for slurries having a total solids content of about 4 wt % of less; screens and drain-screws which may be used for inlet slurries having a total solids content of about 4 wt % to about 18 wt %; and screw presses and plug feeders which may be used for inlet slurries having a total solids content of about 15 wt % to about 40 wt %. Dewatering operations may increase the total solids content of the biomass to about 30 wt % or more, to about 40 wt % or more, to about 50 wt % or more (e.g., from about 30 wt % to about 50 wt % or from about 30 wt % to about 40 wt % total solids). Dewatering produces a effluent slurry 3 (FIG. 1). After dewatering, the dewatered biomass 12 and steam 11 are introduced into a vessel to steam explode the biomass material. Vessels for causing steam explosion of biomass may be referred to as a “pretreatment digester” or simply “digester” or “pretreatment reactor” or simply “reactor” by those of skill in the art and these terms may be used interchangeably herein. The vessel may have any suitable shape (e.g., cylindrical) and may have a vertical or horizontal orientation. Steam is introduced into the vessel at an elevated pressure. Upon discharge from the vessel, the pressure is reduced rapidly which causes a change in the structure of the biomass (e.g., a decrease in the density of the biomass) which allows the cellulose to be more available for downstream enzyme hydrolysis and allows the hemicellulose to be solubilized. The rapid drop in pressure allows a significant portion of the hot condensate to flash off and results in lower temperature and higher solid content of pretreated material.

In some embodiments, the mass ratio of steam 11 to dewatered biomass 12 (based on dry biomass) added to the vessel is at least about 1:6 or, as in other embodiments, at least about 1:4 or at least about 1:1.5. The pressure of steam 11 added to the vessel may be at least about 5 bar, at least about 10 bar or at least about 15 bar. The temperature of steam introduced into the vessel may be from about 150° C. to about 230° C. (e.g., from about 170° C. to about 210° C.).

The temperature within the vessel (and of the biomass after sufficient residence time) may be controlled to be from about 160° C. to about 195° C. by, for example, controlling the pressure of steam introduced into the vessel. In some embodiments and regardless of whether a vertical or horizontal digester is used, the average residence time may be controlled to be between about 1 and about 10 minutes.

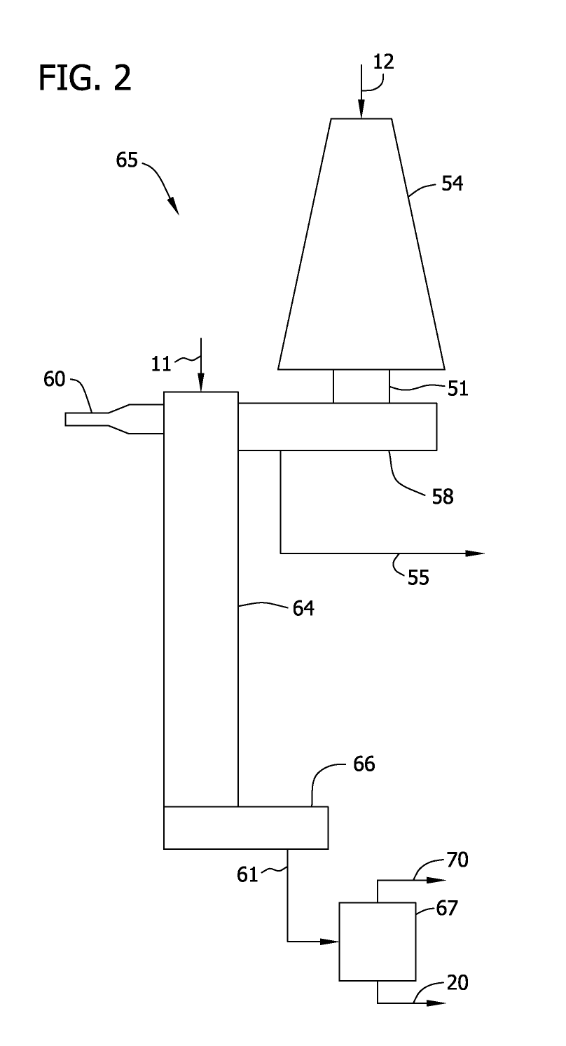

Upon exiting the vessel, the pressure of the biomass is quickly reduced, which causes sudden and vigorous flash of liquid into vapor (often referred to as steam explosion). The steam explosion causes the desired structure change in the biomass (i.e., reduction in particle size and increase in the specific surface area of the biomass). This structure change increases the availability of cellulose to undergo downstream enzymatic hydrolysis. In some embodiments (FIG. 2), the biomass is discharged into a flash vessel 67 that is at a low pressure (e.g., about 0.5 bar to about 3 bar gauge) relative to the steam vessel 64. The pressure difference between the steam vessel and flash vessel may be at least about 5 bar, at least about 9 bar or at least about 12 bar.

Referring now to FIG. 2, a system 65 for causing steam explosion of dewatered biomass material may include a chip silo 54, a pretreatment digester 64 and a plug screw feeder 58 that transfers acid impregnated and dewatered biomass 12 from the silo 54 to the digester 64. In some embodiments, the acid concentration or acidity (e.g., pH) of the liquid effluent 55 discharged from the plug screw feeder 58 (or the effluent 3 (FIG. 1) discharged from upstream dewatering operations) is measured. The acid concentration may be adjusted and thereby controlled by increasing or decreasing the concentration of acid in the acid stream 8 that is used as a source of acid during acid impregnation. In some embodiments, the pH of the liquid effluent 55 or liquid effluent 3 is controlled to be between about 1 and about 2.

The silo 54 is suitably sized to provide sufficient storage capacity to allow acid impregnated and dewatered biomass 12 to be introduced at a relatively constant rate to the pretreatment digester 64. The silo 54 may have a cylindrical shape with a diverging wall (i.e., the diameter of the bottom is larger than the diameter of the top), but may alternatively have another suitable shape. A metering device 51 feeds dewatered biomass 12 from the silo 54 to the plug screw feeder 58. The total solids content of biomass introduced into the digester 64 may be from about 40 wt % to about 60 wt % total solids or, as in other embodiments, from about 45 wt % to about 55 wt % total solids.

While the pretreatment digester 64 is shown in FIG. 2 as being generally vertical, the digester may also be oriented generally horizontally or in other orientations. In addition, while the pretreatment operations have been described with reference to dilute-acid steam pretreatment, the pretreatment operation may involve concentrated acid or steam-only pretreatment processes.

In some embodiments, the pH of the steam-exploded biomass before conditioning (i.e., during steam explosion) is controlled to be less than about 2 or even less than about 1.5. Biomass pH of less than about 2 or even less than about 1.5 may result in relatively high solubilization of hemicellulose.

Biomass may be evenly removed from the bottom of the digester 64 by use of a rotary sweeper (not shown) positioned above a screw conveyor 66 (FIG. 2) which conveys the treated material 61 through a blow valve assembly (not shown) and into a flash vessel 67 in which steam 70 is flashed from the biomass and discharged. The flash vessel 67 may receive material from one pretreatment digester as shown in FIG. 2 or from two or more digesters without limitation.

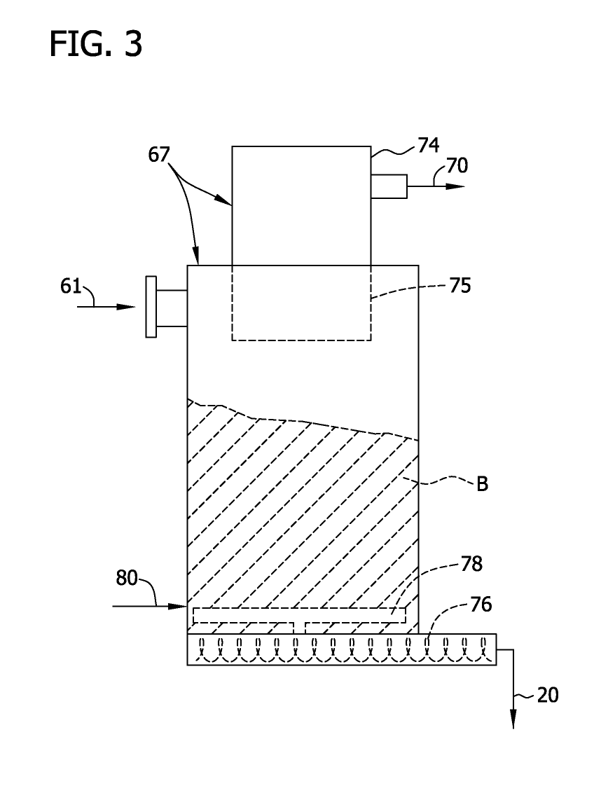

Referring now to FIG. 3, the flash vessel 67 may include a main section 72 that receives biomass. The biomass forms a bed B of biomass within the main section 72. The flash vessel 67 also includes and a stand pipe section 74 through which flashed steam 70 is removed. The stand pipe section 74 has a sufficiently large diameter such that the velocity of the steam 70 removed from the main section 72 is below the terminal velocity (i.e., entrainment velocity) of biomass material 61. Below the stand pipe 74 is a vortex finder 75 with a diameter the same as the diameter of the stand pipe. The vortex finder 75 extends below the entry point of biomass 61 in the main section 72. In some embodiments (not shown), the diameter of the lower section of the vortex finder 75 may be larger than the diameter of its top section to enhance settling of solid particles. The ratio of the diameter of the stand pipe to the diameter of the flash vessel may be from about 1:5 to about 1:1.3 or, as in other embodiments, from about 1:3 to about 1:1.5. The ratio of the height of the vortex finder to the diameter of the flash vessel may be from about 1:2.5 to about 1:1.3 or from about 1:2 to about 1:1.4.

In some embodiments, the average residence time of steam 70 in the flash vessel 67 exceeds the time for at least about 90% of the biomass particles to reach near equilibrium (e.g., within about 5° C., within about 3° C. or within about 1° C. of equilibrium) with the liquor in the vessel. The time for at least about 90% of the biomass particles to reach near equilibrium with the liquor may vary with the size of the particles and the pressure of the flash vessel 67. In some embodiments, the average residence time of flash steam is at least about 1 second, at least about 2 seconds or at least about 4 seconds (e.g., from about 1 second to about 10 seconds or from about 2 seconds to about 6 seconds). The residence time of flash steam may be determined from the flash steam flow in the exhaust pipe connected to the stand pipe 74 and the volume of vapor space in the flash vessel 67 and stand pipe 74 (calculated based on a measured level).

An extraction screw 76 extends into the main section 72 of the flash vessel 67 to remove pretreated biomass material 20. The extraction screw 76 may be a pair (or more) of twin screw conveyors in which the two screws turn in opposite directions. The extraction screw 76 may have mixing paddles or cut flights to mix cooling fluid with the pretreated biomass as described below. An agitator 78 having several blades is located above the screw conveyor to mix the biomass and distribute the biomass onto the extraction screw 76 for removal from the main section 72 of the vessel. A minimum biomass bed height (e.g., about 1 meters) is maintained in the vessel 67 to prevent flash steam from blowing through the bottom outlet of the flash tank through the screw conveyor and the blow valve assembly. The pretreated biomass in the flash vessel 67 may be maintained at a level that ensures sufficient residence time of flash steam and below the inlet of the vortex finder 75 (e.g., at least about 1 meter below the inlet of the vortex finder).

A cooling fluid 80 is added to the vessel 67 to cool the biomass and adjust biomass total solids (and, as in some embodiments, to adjust biomass pH as described below) to prepare the biomass for subsequent enzyme hydrolysis and fermentation. The cooling fluid 80 may be added through one or more injection nozzles (not shown) that extend through the vessel 67. In some embodiments of the present disclosure, the cooling fluid 80 is added to the vessel 67 at a point opposite the bed of biomass (i.e., not above or below the bed). The cooling fluid 80 may be added at or above the agitator 78) and to promote mixing of cooling fluid 80 throughout the biomass. The cooling fluid 80 may be added at the agitator or from about 5 cm to about 50 cm or from about 10 cm to about 30 cm above the agitator blades. The cooling fluid 80 may also be added opposite the outlet of the screw conveyor 76 to promote mixing between the cooling fluid and the biomass before removal from the vessel 67.

In some embodiments, the biomass which initially contacts the cooling fluid is at near equilibrium with the liquor (i.e., liquid associated with the biomass) in the vessel 67 (e.g., within about 5° C., within about 3° C. or within about 1° C.). To ensure that the biomass is near equilibrium with the liquor, the level of biomass in the vessel 67 may be maintained at a sufficient height such that the average residence time of biomass above the entry point of the cooling fluid 80 in the vessel 67 is at least about 10 seconds, at least about 30 seconds or even about 1 minute or more. The residence time may be determined from the measured level of biomass in the flash vessel 67, estimated bulk density of pretreated biomass in the vessel, and the measured mass flow rate of biomass input into the pretreatment digester(s).

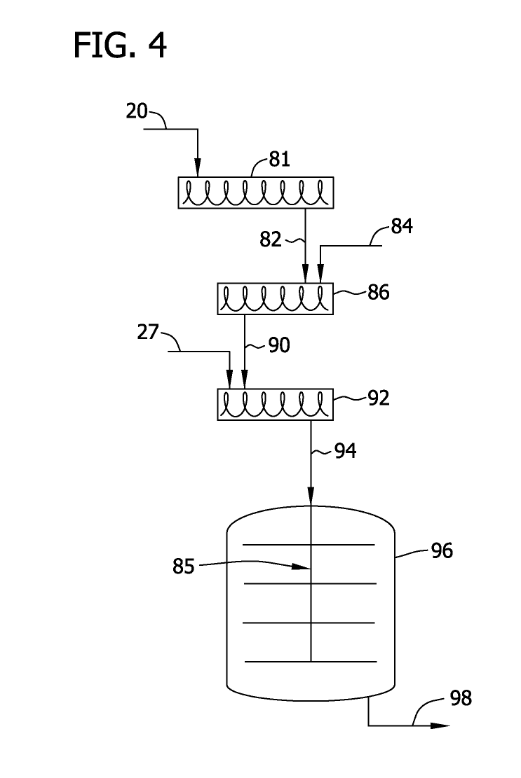

During start-up of the pretreatment process when there is no biomass in the vessel 67, in order to prevent steam from blowing through the bottom of the vessel 67 and entering the mixing screw conveyor 81 (FIG. 4), a sealing device (e.g., a damper or a slide gate valve) (not shown) may be used at the discharge end of the screw conveyor 76. Once the desired level of biomass is established in the flash vessel 67, the damper or valve is opened to begin discharging the pretreated biomass from the vessel into the mixing screw conveyor 81.

In various embodiments, the cooling fluid 80 introduced into the vessel 67 is at a temperature of less than about 25° C. or even less than about 20° C. or less than about 10° C. (e.g., from about 5° C. to about 25° C. or from about 10° C. to about 25° C.). Such temperatures may involve use of a chilling apparatus (not shown) or other suitable methods for cooling the cooling fluid to the desired temperature.

The cooling fluid 80 may reduce the total solids content of the pretreated biomass 20 from a starting solids content (e.g., of material entering the flash vessel 67) in a range of about 35 wt % to about 45 wt % to a total solids content in the range of about 20 wt % to about 35 wt % total solids (e.g., from about 25 wt % to about 30 wt %). The amount of cooling fluid 80 may be adjusted based on the mass flow rate and total solids of biomass added to the pretreatment digester. The temperature and total solids content of the biomass slurry after mixing of the cooling fluid can be measured either in-line using appropriate instrument or of-line of samples taken from the mixing screw conveyor 81 (FIG. 4).

The cooling fluid 80 may contain an alkali (i.e., base) to adjust the pH of the pretreated biomass (in which case the cooling fluid 80 may be referred to herein as an “alkaline solution”). The alkali may be added to the cooling fluid by mixing process water and a concentrated alkali such as, for example, ammonia, aqueous ammonia, or alkali or alkaline earth metal hydroxide (e.g., NaOH, KOH). In some embodiments, different alkali solution is added at different process points. For example, NaOH may be added to the flash vessel 67 and aqueous ammonia may be added to the neutralization mixer as described below.

Cooling fluid 80 may be used to adjust pH in the vessel 67 (rather than adjustment occurring solely downstream) to provide adequate contact time for the fluid to neutralize the biomass before enzyme addition. In some embodiments of the present disclosure, use of alkaline cooling fluid 80 may provide coarse adjustment of biomass pH and fine adjustment may occur downstream of the flash vessel 67.

Alkaline cooling fluid may be cooled after addition of alkali or the process water may be cooled before mixing. The alkali and process water may be mixed by any suitable method such as by use of a static in-line mixer. Adjustment of biomass pH may be controlled by adjusting the concentration of alkali in the cooling fluid 80 (and, optionally, the process stream 84 described below). The temperature of the biomass slurry may controlled by adjusting the temperature of chilled fluid 80 and the amount of fluid added. The total solids content of the resulting biomass slurry may be controlled by adjusting the amount of fluid 80 added and, to a lesser extent, the operating pressure of the flash tank (i.e., at lower operating pressures more condensate flashes off).

It should be further noted that while the processes described herein may be described with reference to dilute acid pretreatment operations (e.g., contacting the biomass with a source of acid containing less than about 5 wt % acid), the processes may also apply to alkali pretreatment and/or auto-hydrolysis pretreatment operations unless stated otherwise. In embodiments in which alkali pretreatment is used, the cooling fluid 80 may contain acid.

Upon discharge from the vessel 67, the pretreated biomass 20 is introduced into a mixing screw conveyor 81 which provides further mixing of biomass to uniformly cool the biomass. The average residence time of biomass between contact with cooling fluid 80 (FIG. 3) and discharge from the mixing screw conveyor 81 may be at least about 30 seconds, at least about 1 minute or at least about 2 minutes or more to provide adequate blending of cooling fluid and biomass.

The mixing screw conveyor 81 may be water jacketed to provide further cooling. In some embodiments, the temperature of the discharge slurry 82 (and in some embodiments of the pretreated biomass 20) may be less than about 90° C., less than about 80° C., less than about 70° C., less than about 60° C. or even less than about 50° C. (e.g., from about 40° C. to about 90° C. or from about 40° C. to about 70° C.).

The discharge 82 from the mixing screw conveyor 81 is introduced into a neutralization mixer 86. An alkaline process stream 84 (which may be cooled as described below) is added to the mixer 86 to provide the final adjustment of pH and total solids content (and, in some embodiments, temperature) of the pretreated biomass slurry before enzymatic hydrolysis. The pH adjustment in the mixer 86 may be a “fine” adjustment (e.g., 0.1 to 0.4 pH adjustment) with the crude adjustment occurring upstream in the flash vessel 67. The amount of pH adjustment may be controlled by changing the concentration of alkali in the alkaline process stream 84. After final pH adjustment, the pH of the neutralized biomass 90 may be, for example, from about 4.0 to about 5.5 or from about 4.8 to about 5.2 depending on the particular enzyme used for liquefaction.

In some embodiments, the alkaline process stream 84 is cooled such as to a temperature less than about 25° C., less than about 20° C. or less than about 15° C. (e.g., from about 5° C. to about 25° C. or from about 10° C. to about 20° C.). The neutralization mixer 86 may be water jacketed to provide cooling. The mixer 86 may be any suitable mixing apparatus and may be a dynamic mixer such as a paddle mixer, pug mill mixer or helical mixer. The alkaline process stream 84 also provides final adjustment of the total solids content of the conditioned biomass. The amount of process stream 84 mixed with the biomass in the mixer may be adjusted based on total solids measurements made downstream of the mixer 86 (e.g., in the discharge) and upstream of the liquefaction bioreactor 96. Total solids content may be determined by sampling the material and measuring the mass of the sample before and after evaporating the moisture by heating (e.g., by infrared (IR) moisture balances). The total solids content may also be measured by near infrared (NIR) moisture analyzers or portable (e.g., handheld) moisture meters.

It should be noted that the conditioning processes described herein are exemplary and additional process steps and equipment may be used and/or steps or equipment may be eliminated or substituted for other processes or equipment. For example, conditioning may include use of one or more disk refiners or disintegrators (not shown).

Conditioned biomass 90 is introduced into an enzyme mixer 92. The mixer 92 may be a dynamic mixer such as a paddle mixer, pug mill mixer or other suitable mixing apparatus. Enzyme 27 is added to the mixer (e.g., enzyme dispersed through a liquid medium such as water) to begin enzymatic hydrolysis of the conditioned feedstock. In some embodiments, an enzyme 27 is added to cause downstream liquefaction of the biomass in which the conditioned biomass transitions from a high viscosity slurry to a pumpable low viscosity slurry. Suitable enzymes include, for example, cellulase, hemicellulase, pectinase, and lignin degrading enzyme.

After addition of enzyme, the enzyme-containing pretreated biomass slurry 94 is introduced into a liquefaction bioreactor 96 to partially hydrolyze the biomass thereby reducing the viscosity of the biomass (e.g., from a starting viscosity of about 20,000 cP or more to a reduced viscosity of about 5,000 cP or less) to allow it to be pumped and processed in downstream saccharification operations. The liquefaction bioreactor may be a plug-flow reactor with a suitable height-to-diameter ratio (e.g., of at least 2:1, at least about 3:1 or even about 4:1 or more). The average residence time through the bioreactor 96 may be at least about 10 minutes, at least about 15 minutes, at least about 30 minutes or at least about 45 minutes (e.g., from about 10 to about 90 minutes or from about 15 to about 60 minutes).

The bioreactor 96 includes an agitator 85 having multiple impellers that creates one or more mixing zones (e.g., 2, 3, 4 or 5 or more mixing zones) in the reactor. The top-most impeller may be positioned near the surface of the slurry to disperse the biomass across the reactor 96. The hydraulic residence time in each mixing zone may be, for example, about 1 to about 10 minutes (e.g., about 1 to about 3 minutes). The height of each mixing zone may be from about 0.6 to about 1.2 meters. The mixing zones may be separated by an average hydraulic residence time of from about 2 to about 10 minutes. The impellers may be evenly spaced or unevenly spaced with the spacing being less near the upper portions of the reactor where slurry viscosity is relatively higher. Each impeller may be sized and shaped to provide a suitable mixing zone height. The impeller design and rotational speed of the agitator may be selected to provide radial mixing of the slurry with minimal vertical pumping action.

The particular bioreactor dimensions, residence times and impeller designs described above are exemplary and any suitable dimensions, residence times and impeller designs may be used without limitation unless stated otherwise herein.

In some embodiments of the present disclosure, pH of the partially hydrolyzed pretreated biomass is measured in the liquefaction bioreactor 96. The measured pH may be used to provide fine pH control by adjusting one or both of (1) the amount of alkaline solution 84 introduced into the neutralization mixer 86 and (2) the concentration of alkali in the alkaline solution 84. Alternatively or in addition, the measure pH may be used to provide coarse pH control by adjusting one or both of (1) the amount of alkaline solution (e.g., cooling fluid) introduced into the flash vessel 67 (FIG. 3) and (2) the concentration of alkali in the alkaline solution added to the flash vessel based on the measured pH of the partially hydrolyzed pretreated biomass in the liquefaction bioreactor. The pH may be measured at any point (middle, top or bottom of the slurry) in the bioreactor 96 including at the discharge of the liquefied slurry 98. In-line pH or conductivity meters may be used to continually monitor the pH of the slurry in the bioreactor 96 or the pH of samples of slurry taken from the bioreactor may be measured by trained technicians or laboratory personnel. In some embodiments, the measured pH is used, at least partially, to adjust the concentration of alkali in the cooling fluid 80 (FIG. 3).

As an alternative to measurement of pH in the bioreactor 96, the pH of the conditioned biomass 90 or enzyme-containing biomass 94 may be measured for pH feedback control in the neutralization mixer 86.

In some embodiments of the present disclosure, pretreated biomass downstream of the digester 64 (FIG. 2) and upstream of the bioreactor 96 (FIG. 4) is analyzed to provide feedback information for the pretreatment process. For example, one or more of the following may be determined for feedback control of the pretreatment operations: pH (before conditioning), total solids, liquid fraction composition (e.g., sugars, degradation products of carbohydrates and lignin such as furfural, hydroxymethyl furfural, acetic acid, phenolic compounds) and solid fraction composition (e.g., glucan, xylan, lignin (including pseudo-lignin). The water-insoluble fraction of pretreated biomass may be monitored (intermittently or continuously, in-line or off-line) using near-infrared (NIR) or Fourier Transform NIR (FT-NIR) spectroscopy with multivariate analysis or by other suitable methods.

After liquefaction, the partially hydrolyzed slurry 98 is subjected to additional hydrolysis operations such as saccharification operations. Additional enzymes 27 (FIG. 1) such as cellulase, xylanase, β-xylosidase, acetyl esterase, and α-glucuronidase, endo- and exo-glucannase, cellobiase, lignin degrading enzymes and combinations of these enzymes may be added. Enzymatic hydrolysis may involve separation steps in which C5 sugars are separated from cellulose containing streams and/or in which lignin is separated from the biomass. Any suitable method for hydrolysis of hemicellulose and cellulose which results in fermentable (C5 and/or C6 sugars) may be used in accordance with the present disclosure without limitation.

After production of simple sugars, the sugars 40 (C5 and/or C6 sugars) may be fermented to produce ethanol. In this regard, fermentation of C5 and C6 sugars may be conducted together or separately (e.g., sequentially or in parallel in embodiments in which the C5 and C6 sugars are separated). Any suitable yeast 36 may be used depending on the sugar content of the fermentable stream. Saccharification and fermentation may, at least partially, be achieved in the same vessel or these operations may be performed separately.

Fermentation product stream 42 is subjected to various ethanol recovery steps (e.g., distillation and molecular sieving) to recover ethanol 50. A stillage stream 52 may be removed from the distillation bottoms which may be processed to produce various co-products such as dried distillers biomass or dried distillers biomass with solubles.

It should be noted that the process for producing ethanol from biomass feedstock shown in FIG. 1 and as described herein is simplified for clarity and commercial processes may include additional processing steps, equipment, process recycles and the like. Exemplary ethanol production based on biomass feedstock is also described in U.S. Pat. Pub. No. 2012/0006320, which is incorporated herein by reference for all relevant and consistent purposes.

Compared to traditional methods, the methods described above have several advantages. By introducing cooling fluid 80 (FIG. 3) to the flash vessel 67 opposite the bed of biomass (e.g., such that the residence time of biomass above the addition point of cooling fluid is at least about 10 seconds or at least about 1 minute), the biomass can reach the equilibrium temperature of the liquor in the flash vessel before contacting the cooling fluid. This promotes cooling that results from evaporation of condensate. Heat from the biomass may transfer to the liquor and continued flashing of water from the liquor may be achieved, which results in cooling of the biomass and reduction of the water content of the resulting biomass material. This allows less cooling liquid to be used to achieve the target temperature and total solid content of the biomass slurry relative to conventional methods.

Further, in embodiments in which a second alkali process stream (e.g., process stream 84) is used, fine adjustment of pH, temperature and/or total solids content of the resulting biomass slurry may be achieved. The second process stream also provides process flexibility (e.g., different types of alkali may be used in cooling fluid 80 and process stream 84). By using chilled cooling fluid 80 (e.g., below about 25° C.) less cooling fluid may be used to cool the biomass to the target temperature which allows a relatively high total solids content of biomass slurry to be maintained. By measuring the pH in the bioreactor 96 (FIG. 4) and using the measured pH for control of the amount or concentration of alkaline process stream 84, improved control may be realized as the lower viscosity slurry in the bioreactor may provide a more reliable and accurate pH measurement.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

1. A method for conditioning pretreated biomass, the method comprising:
discharging biomass from a pretreatment digester to a flash vessel to flash vapor from the biomass, the biomass forming a bed in the flash vessel; and
introducing a cooling fluid into the flash vessel to cool the biomass, the cooling fluid having a temperature less than about 25° C.

2. The method as set forth in claim 1 wherein the cooling fluid is introduced into the flash vessel at a point opposite the bed.

3. The method as set forth in claim 1 wherein the flash vessel comprises an agitator for mixing and distributing the biomass onto an extraction screw for removing biomass from the flash vessel, the cooling fluid being added to the flash vessel above the agitator.

4. The method as set forth in claim 1 wherein the cooling fluid has a temperature less than about 20° C.

5. The method as set forth in claim 1 wherein the cooling fluid has a temperature less than about 15° C.

6. The method as set forth in claim 1 wherein the cooling fluid contains an alkali for increasing the pH of the biomass.

7. The method as set forth in claim 6 wherein the concentration of alkali in the cooling fluid is adjusted based on at least one of (1) the rate of addition of biomass into the pretreatment digester and (2) a measured pH of biomass downstream of the flash vessel.

8. The method as set forth in claim 1 wherein the rate of cooling fluid introduced into the flash vessel is adjusted based on at least one of (1) the rate of addition of biomass into the pretreatment digester and (2) a measured total solids of biomass downstream of the flash vessel.

9. The method as set forth in claim 1 further comprising:
discharging the biomass from the flash vessel;
introducing the discharged biomass into a neutralization mixer; and
introducing an alkaline process stream into the neutralization mixer, the alkaline process stream having a temperature less than about 25° C.

10. The method as set forth in claim 1 wherein the residence time of biomass above the entry point of the cooling fluid in the vessel is at least about 10 seconds.

11. The method as set forth in claim 1 wherein the residence time of biomass above the entry point of the cooling fluid in the vessel is at least about 30 seconds.

12. The method as set forth in claim 1 wherein the residence time of biomass above the entry point of the cooling fluid in the vessel is at least about 1 minute.

13. The method as set forth in claim 1 further comprising:
contacting a biomass feedstock with an aqueous acid solution to prepare an acid-impregnated biomass stream, the aqueous acid solution having an acid concentration of less than about 5 wt %; and
introducing the acid-impregnated biomass stream into the pretreatment digester.

14. The method as set forth in claim 1 comprising discharging pretreated biomass from the flash vessel, the temperature of the pretreated biomass being about 90° C. or less.

15. The method as set forth in claim 1 comprising discharging pretreated biomass from the flash vessel, the temperature of the pretreated biomass being about 70° C. or less.

16. A method for conditioning pretreated biomass, the method comprising:
discharging pretreated biomass from a pretreatment digester;
introducing the pretreated biomass discharged from the digester into a neutralization mixer;
introducing an alkaline solution into the neutralization mixer to adjust the pH of the biomass;
adding enzyme to the pH-adjusted pretreated biomass;
introducing the enzyme-containing pretreated biomass into a liquefaction bioreactor to partially hydrolyze the pretreated biomass;
measuring the pH of the partially hydrolyzed pretreated biomass in the liquefaction bioreactor; and
adjusting at least one of (1) the amount of alkaline solution introduced into the neutralization mixer and (2) the concentration of alkali in the alkaline solution based on the measured pH of the partially hydrolyzed pretreated biomass in the liquefaction bioreactor.

17. The method as set forth in claim 16 wherein the concentration of alkali in the alkaline solution is adjusted based on the measured pH of the partially hydrolyzed pretreated biomass in the liquefaction bioreactor.

18. The method as set forth in claim 16 wherein the amount of alkaline solution introduced into the neutralization mixer is adjusted based on the total solids content of the pH-adjusted pretreated biomass.

19. The method as set forth in claim 16 wherein the pH of the partially hydrolyzed pretreated biomass is measured at the discharge of the liquefaction bioreactor.

20. The method as set forth in claim 16 further comprising:
contacting a biomass feedstock with an aqueous acid solution to prepare an acid-impregnated biomass stream, the aqueous acid solution having an acid concentration of less than about 5 wt %; and
introducing the acid-impregnated biomass stream into the pretreatment digester.

21. A method for conditioning pretreated biomass, the method comprising:
discharging pretreated biomass from a pretreatment digester into a flash vessel;
introducing an alkaline solution into the flash vessel to adjust the pH of the biomass;
adding enzyme to the pH-adjusted pretreated biomass;
introducing the enzyme-containing pretreated biomass into a liquefaction bioreactor to partially hydrolyze the pretreated biomass;
measuring the pH of the partially hydrolyzed pretreated biomass in the liquefaction bioreactor; and
adjusting at least one of (1) the amount of alkaline solution introduced into the flash vessel and (2) the concentration of alkali in the alkaline solution based on the measured pH of the partially hydrolyzed pretreated biomass in the liquefaction bioreactor.

22. The method as set forth in claim 21 wherein the concentration of alkali in the alkaline solution is adjusted based on the measured pH of the partially hydrolyzed pretreated biomass in the liquefaction bioreactor.

23. The method as set forth in claim 21 wherein the amount of alkaline solution introduced into the flash vessel is adjusted based on the total solids content of the pH-adjusted pretreated biomass.

24. The method as set forth in claim 21 wherein the pH of the partially hydrolyzed pretreated biomass is measured at the discharge of the liquefaction bioreactor.

25. The method as set forth in claim 21 further comprising:
contacting a biomass feedstock with an aqueous acid solution to prepare an acid-impregnated biomass stream, the aqueous acid solution having an acid concentration of less than about 5 wt %; and
introducing the acid-impregnated biomass stream into the pretreatment digester.
